(19) United States
(12) Patent Application Publication
(10) Pub. No.: US 2024/0099562 A1
(43) Pub. Date: Mar. 28, 2024

(54) HYBRID COUPLING MECHANISM FOR AN ENDOSCOPE SYSTEM

(71) Applicant: Boston Scientific Scimed, Inc., Maple Grove, MN (US)

(72) Inventors: Ryan V. Wales, Northborough, MA (US); Ryan Vincent William Pollock, Leominster, MA (US); Colby Harris, Norfolk, MA (US); Brittany Elizabeth Reed, Brighton, MA (US)

(73) Assignee: Boston Scientific Scimed, Inc., Maple Grove, MN (US)

(21) Appl. No.: 18/469,693

(22) Filed: Sep. 19, 2023

Related U.S. Application Data
(60) Provisional application No. 63/410,159, filed on Sep. 26, 2022.

(51) Int. Cl. A61B 1/00; A61B 1/015; A61B 1/12

(52) U.S. Cl. CPC A61B 1/00128; A61B 1/00119; A61B 1/015; A61B 1/126

(57) ABSTRACT

An example medical device includes a container and tube set arranged and configured to couple to an endoscope, a first gas supply, and a second gas supply for use in an endoscopic procedure. The container and tube set includes a container having an interior volume configured to contain a fluid, a gas supply tube having a first end and a second end in fluid communication with the interior volume of the container, and a coupling mechanism having a first end and a second end, wherein the first end of the coupling mechanism is configured to engage with the second end of the gas supply tube and the second end of the coupling mechanism is configured to engage with both the first gas supply and the second gas supply interchangeably.

### CROSS-REFERENCE TO RELATED APPLICATIONS

This application claims the benefit of U.S. Provisional Patent Application Ser. No. 63/410,159 filed on Sep. 26, 2022, the disclosure of which is incorporated herein by reference.

### TECHNICAL FIELD

The present disclosure pertains to medical devices, and methods for manufacturing medical devices. More particularly, the present disclosure pertains to coupling mechanisms to supply fluid and/or gas to an endoscope.

### BACKGROUND

Conventionally, endoscope devices have been widely used for performing diagnostic and/or therapeutic treatments. Endoscope devices are often coupled to additional devices, such as, but not limited to, processors, light sources, water sources, gas sources, etc. For example, water is supplied to the endoscope for irrigation and lens washing while air/gas is supplied for insufflation of the working lumen. As new endoscope technologies emerge, the new technologies may not be backwards compatible with older technology. This may necessitate more than one connector or types of connectors be available for coupling the additional devices to the endoscope. However, having multiple pieces of tubing, hoses, couplers, etc. can be cumbersome within the procedure suite. It is with these considerations in mind that the improvements of the present disclosure may be useful.

### BRIEF SUMMARY

This disclosure provides design, material, manufacturing method, and use alternatives for medical devices. An example of a medical device may include a container and tube set arranged and configured to couple to an endoscope, a first gas supply, and a second gas supply for use in an endoscopic procedure. The container and tube set may include a container having an interior volume configured to contain a fluid, a gas supply tube including a first end, a second end, and a first lumen extending therethrough, wherein the first end of the gas supply tube terminates at or in the container and the first lumen is in fluid communication with the interior volume of the container, a coupling mechanism having a first end and a second end, wherein the first end of the coupling mechanism may be configured to engage with the second end of the gas supply tube and the second end of the coupling mechanism may be configured to engage with both the first gas supply and the second gas supply interchangeably, and a water supply tube including a first end, a second end, and a second lumen extending therethrough, wherein the first end of the water supply tube terminates at or in a bottom portion of the container, the second lumen is in fluid communication with the interior volume of the container, and the second end of the water supply tube is positioned external to the container.

Alternatively or additionally to any of the embodiments above, the container and tube set may include a split flow junction at the second end of the water supply tube wherein the second lumen of the water supply tube may be in fluid communication with both a third lumen of a lens wash tube and a fourth lumen of an irrigation supply tube.

Alternatively or additionally to any of the embodiments above, the second end of the coupling mechanism may include a plurality of ridges on the outer surface of the coupling mechanism.

Alternatively or additionally to any of the embodiments above, an annular seal comprising at least one of the plurality of ridges may be positioned between two others of the plurality of ridges.

Alternatively or additionally to any of the embodiments above, the plurality of ridges may include at least a first ridge of the plurality of ridges having a size and shape configured to engage with the first gas supply, such that a seal is formed between the coupling mechanism and the first gas supply and at least a second ridge of the plurality of ridges having a size and shape configured to engage with the second gas supply, such that a seal is formed between the coupling mechanism and the second gas supply.

Alternatively or additionally to any of the embodiments above, the first end of the coupling mechanism may include a hose barb mechanism.

Alternatively or additionally to any of the embodiments above, the coupling mechanism may be a single, monolithic structure.

Alternatively or additionally to any of the embodiments above, the second end of the coupling mechanism may be shaped to facilitate an interference fit between the coupling mechanism and the first gas supply and the second gas supply interchangeably.

Alternatively or additionally to any of the embodiments above, the first gas supply may be a processor capital configured to pump air through the gas supply tube and wherein the second gas supply is a carbon dioxide (CO₂) source configured to pump CO₂ through the gas supply tube.

Another example of a medical device may include a coupling mechanism for an endoscope system. The coupling mechanism may include a substantially tubular body having an outer surface, and inner surface, a first end, and a second end, and a lumen extending through the tubular body from the first end to the second end. The first end may be configured to engage with a gas supply tube and the second end may be configured to engage with both a first gas supply and a second gas supply interchangeably, and the gas supply tube may be configured to engage with a container configured to contain a fluid.

Alternatively or additionally to any of the embodiments above, the second end of the coupling mechanism may include a plurality of ridges on the outer surface of the coupling mechanism.

Alternatively or additionally to any of the embodiments above, the first end of the coupling mechanism may include a hose barb mechanism.

Alternatively or additionally to any of the embodiments above, an annular seal comprising at least one of the plurality of ridges may be positioned between two others of the plurality of ridges.

Alternatively or additionally to any of the embodiments above, the plurality of ridges may include at least a first ridge of the plurality of ridges having a size and shape configured to engage with the first gas supply, such that a seal is formed between the coupling mechanism and the first gas supply and at least a second ridge of the plurality of ridges having a size and shape configured to engage with the second gas supply, such that a seal is formed between the coupling mechanism and the second gas supply.

Alternatively or additionally to any of the embodiments above, the first gas supply may be a processor capital configured to pump air through the gas supply tube.

Alternatively or additionally to any of the embodiments above, the second gas supply may be a carbon dioxide (CO₂) source configured to pump CO₂ through the first gas supply tube.

Another example of a medical device may include a coupling mechanism for an endoscope system. The coupling mechanism may include a substantially tubular body having an outer surface, and inner surface, a first end, and a second end. A lumen may extend through the tubular body from the first end to the second end, a hose barb mechanism may be positioned at the first end of the tubular body, and a plurality of ridges may be positioned at an outer surface of the second end of the tubular body. An annular seal may be positioned within the plurality of ridges, the first end may be configured to engage with a second end of a gas supply tube and the second end may be configured to engage with both a first gas supply and a second gas supply interchangeably, and a first end of the gas supply tube may be configured to engage with a container configured to contain a fluid.

Alternatively or additionally to any of the embodiments above, the first gas supply may be a processor capital configured to pump air through the gas supply tube, and the second gas supply may be a carbon dioxide (CO₂) source configured to pump CO₂ through the gas supply tube.

Alternatively or additionally to any of the embodiments above, the plurality of ridges may include at least a first ridge of the plurality of ridges having a size and shape configured to engage with the first gas supply, such that a seal is formed between the coupling mechanism and the first gas supply and at least a second ridge of the plurality of ridges having a size and shape configured to engage with the second gas supply, such that a seal is formed between the coupling mechanism and the second gas supply.

Alternatively or additionally to any of the embodiments above, the second end of the coupling mechanism may be shaped to facilitate an interference fit between the coupling mechanism and the first gas supply and the second gas supply interchangeably.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 illustrates an exemplary container and tube set including a container, a gas supply tube, a lens wash tube, and an irrigation supply tube;

FIG. 2A illustrates a cross-section view of the gas supply tube as in FIG. 1, taken at line 2A-2A;

FIG. 2B illustrates a cross-section view of a water supply tube as in FIG. 1, taken at line 2B-2B;

FIG. 2C illustrates a cross-section view of the lens wash tube as in FIG. 1, taken at line 2C-2C;

FIG. 2D illustrates a cross-section view of the irrigation supply tube as in FIG. 1, taken at line 2D-2D;

FIG. 3 illustrates an exemplary container and tube set coupled to an exemplary first gas supply;

FIG. 4 illustrates an exemplary endoscope system including a container and tube set coupled to an exemplary second gas supply, a lens wash tube, and an irrigation supply tube;

FIG. 5 illustrates a perspective view of an exemplary coupling mechanism;

FIG. 6 illustrates an exemplary coupling mechanism coupled to an exemplary outlet of a first gas supply;

FIG. 7 illustrates a cross-section view of the exemplary coupling mechanism coupled to the exemplary outlet of the first gas supply of FIG. 6, taken at line 7-7;

FIG. 8 illustrates an exemplary coupling mechanism coupled to an exemplary outlet of a second gas supply;

FIG. 9 illustrates a cross-section view of the exemplary coupling mechanism coupled to the exemplary outlet of the second gas supply of FIG. 8, taken at line 9-9;

FIG. 10 illustrates an exemplary coupling mechanism coupled to an exemplary outlet of a gas supply; and

FIG. 11 illustrates a cross-section view of the exemplary coupling mechanism coupled to the exemplary outlet of the gas supply of FIG. 10, taken at line 11-11.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the term “about” may include numbers that are rounded to the nearest significant figure.

The recitation of numerical ranges by endpoints includes all numbers within that range (e.g., 1 to 5 includes, 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in this specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used in connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

An endoscope is used in performing diagnostic and/or therapeutic treatments by inserting an elongated shaft of the endoscope into a subject to observe a part to be examined within a body cavity of the subject and, if necessary, inserting a treatment instrument/tool into a working channel in the elongated shaft of the endoscope. Such endoscopes or endoscope systems may include a fluid/lens wash capability, or the like, configured to feed fluid such as gas (e.g., air, CO2) to an end of the endoscope for insufflating the inside of the subject at a target site. Lens wash features may provide sterilized water at relatively high pressure to spray across and clear debris from a camera lens of the endoscope. In order to rinse the target site of the subject, separate from the air/water feed capability, endoscopes or endoscope systems may have an irrigation capability that provides lower pressure, higher volume water, supplied via a pump (e.g., peristaltic pump) to the target site in order to clear the field of view for observation and treatment. A water source for lens wash and/or irrigation features may include one or more fluid reservoirs having tubing and cap assemblies that create a plumbing circuit in connection with the endoscope channels, valving, and/or connectors to accomplish the gas and water functions described.

Such tubing and cap assemblies may be available in various configurations, which may include a water bottle, a cap fitted for the specific bottle, and an array of tubing that is extendable through openings in the cap. The tubing typically is arranged to accommodate a specific configuration of endoscope fittings and valving, which does not tend to be modular or optional.

During endoscopic procedures, as mentioned, a clinician will utilize air, irrigation, and a lens wash function. To enable these features, either compressed air from a processor or carbon dioxide (CO₂) from a CO₂ source is used to insufflate a working lumen or increase pressure within a water container (e.g., a water bottle), forcing the water within the container to irrigate the lumen or wash the lens on the endoscope. In such cases, coupling the air and/or the CO₂ to the water container may require two different types of coupling mechanisms, one for air and one for CO₂. This disclosure discusses various coupling mechanisms that addresses the above noted issues and/or other issues with existing connectors.

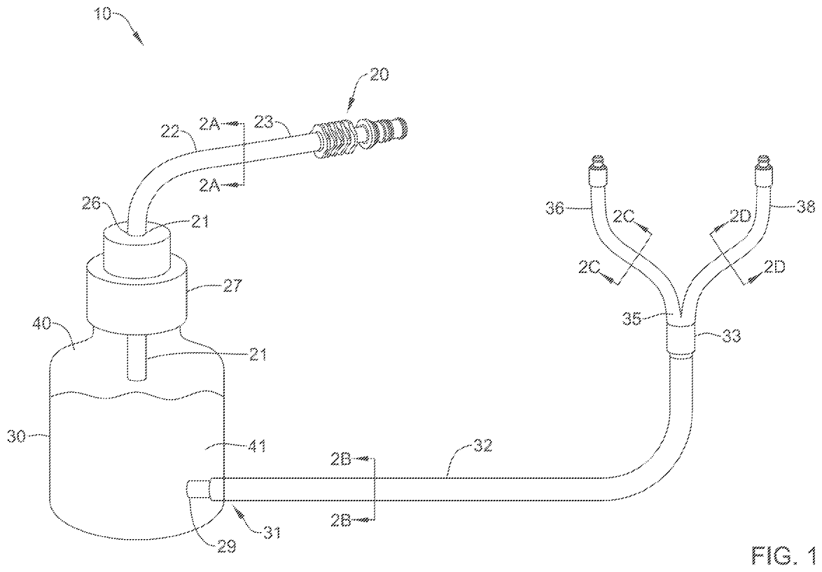

FIG. 1 illustrates an exemplary container and tube set 10 including a container 30, a gas supply tube 22, a lens wash tube 36, and an irrigation supply tube 38. In some cases, the container 30 may be a water reservoir (e.g., a water bottle) and may include an interior volume 40 configured to contain a fluid 41 (e.g., water). The gas supply tube 22 may include a first end 21 and a second end 23 and a first lumen extending therethrough (not shown in FIG. 1). The first end 21 of the gas supply tube 22 may terminate at or within the container 30 such that the first lumen is in fluid communication with the interior volume 40 of the container 30. In some cases, the gas supply tube 22 may pass through an opening 26 in the cap 27 and extend within and into the interior volume 40 of the container 30. In some cases, the opening 26 may include an air gap. In some cases, the opening 26 may include a rubber seal that the gas supply tube 22 may be configured to pass through. In some cases, the first end 21 of the gas supply tube 22 may be detachable, and may engage with the cap 27 of the container 30, for example, using a luer lock system. In some cases, the gas supply tube 22 and the cap 27 may be a single, monolithic piece.

The second end 23 of the gas supply tube 22 may be configured to engage with a coupling mechanism 20. In some cases, the second end 23 of the gas supply tube 22 may engage with the coupling mechanism 20, for example, via a hose barb mechanism. In some cases, the second end 23 of the gas supply tube 22 may engage with the coupling mechanism 20 via interference fit, a luer lock system, a luer slip system, or any other suitable type of engagement.

A water supply tube 32 may include a first end 31, a second end 33, and a second lumen extending therethrough (not shown in FIG. 1). The water supply tube 32 may be configured to terminate at or in a bottom portion of the container 30 such that the second lumen is in fluid communication with the interior volume 40 of the container 30. In some cases, when the water supply tube 32 terminates at the bottom portion of the container 30, the water supply tube 32 may include an inner tube 29 that may extend within and into the interior volume 40 of the container 30, which may contain the fluid 41. For example, the water supply tube 32 may act as a lens wash supply tube and the inner tube 29 may act as an irrigation supply tube, or the water supply tube 32 may act as an irrigation supply tube and the inner tube 29 may act as a lens wash supply tube, among other possibilities. In some cases, the water supply tube 32 may pass through an opening in the bottom portion of the container 30 and extend within and into the interior volume 40 of the container 30. In some cases, the opening in the bottom portion of the container may include a rubber seal that the water supply tube 32 may be configured to pass through. In some cases, the first end 31 of the water supply tube 32 may be detachable, and may engage with the container 30, for example, using a luer lock system, and the inner tube 29 may then extend through the opening in the bottom portion of the container 30 and into the interior volume 40 of the container 30. In some cases, the water supply tube 32 and the container 30 may be a single, monolithic piece.

The second end 33 of the water supply tube 32 may include a split flow junction 35 wherein the second lumen of the water supply tube 32 may be in communication with a third lumen of the lens wash tube 36 and a fourth lumen of the irrigation supply tube 38. While it is shown that the lens wash tube 36 and the irrigation supply tube 38 may source the fluid 41 (e.g., water) from the same water supply tube 32, it may be contemplated that the lens wash tube 36 may source the fluid 41 from the water supply tube 32 and the irrigation supply tube 38 may source a fluid from another fluid source.

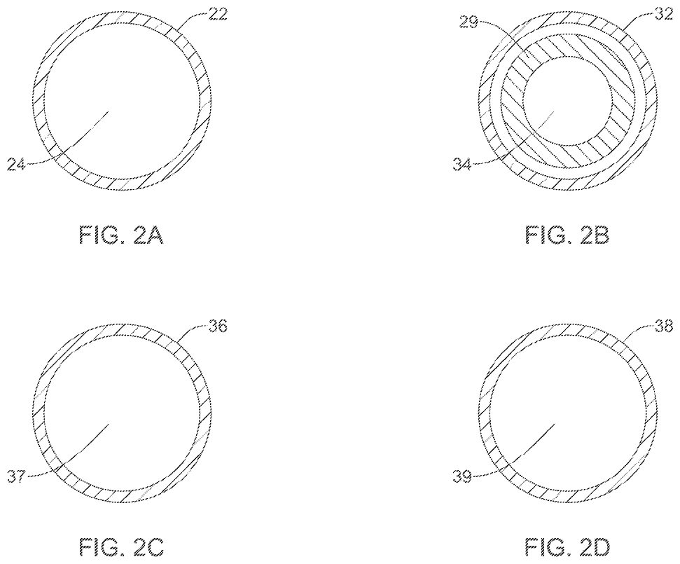

FIG. 2A illustrates a cross-section view of the gas supply tube 22 as in FIG. 1, taken at line 2A-2A. As shown in FIG. 2A, the gas supply tube 22 is a singular tube. In some cases, the gas supply tube 22 may include an outer diameter of 2 mm (millimeters) to 7 mm. FIG. 2B illustrates a cross-section view of the water supply tube 32 as in FIG. 1, taken at line 2B-2B. As shown in FIG. 2B, the water supply tube 32 may include the inner tube 29 and a second lumen 34 extending therethrough. While it is shown that the water supply tube 32 includes the inner tube 29, it may be contemplated that the water supply tube 32 is a singular tube and does not include the inner tube 29. In some cases, the water supply tube 32 may include an outer diameter of 2 mm to 7 mm. FIG. 2C illustrates a cross-section view of the lens wash tube 36 as in FIG. 1, taken at line 2C-2C, and FIG. 2D illustrates a cross-section view of the irrigation supply tube 38 as in FIG. 1, taken at line 2D-2D. As shown in FIG. 2C, the lens wash tube may include a third lumen 37 extending therethrough. In some cases, the lens wash tube 36 may include an outer diameter of 2 mm to 7 mm. As shown in FIG. 2D, the irrigation supply tube 38 may include a fourth lumen 39 extending therethrough. In some cases, the irrigation supply tube 38 may include an outer diameter of 2 mm to 7 mm.

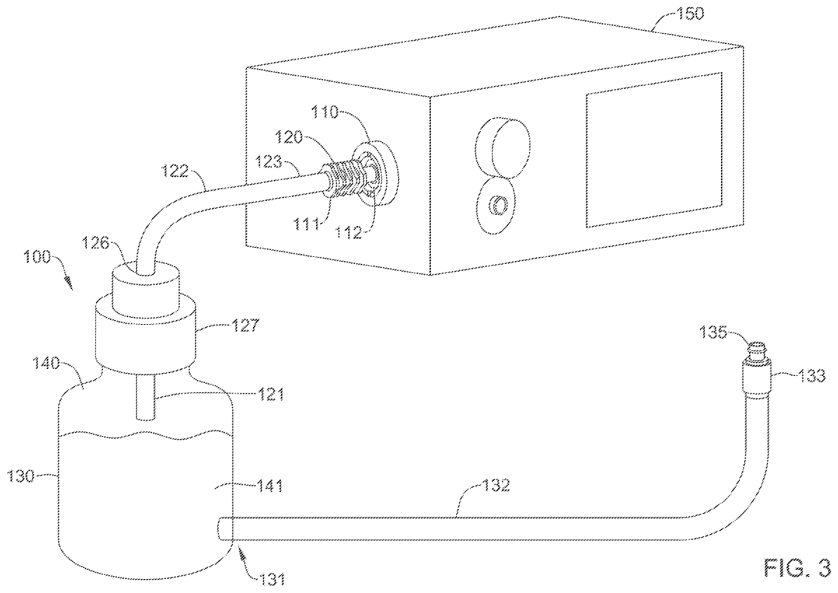

FIG. 3 illustrates an exemplary container and tube set 100 coupled to an exemplary first gas supply 150. The container and tube set 100 may be considered as an example of the container and tube set 10, as in FIG. 1. In some cases, the first gas supply 150 may be a processor capital configured to pump air through a gas supply tube 122 to a container 130. As shown in FIG. 3, the gas supply tube 122 may include a first end 121 and a second end 123. While not shown, the gas supply tube 122 may include a first lumen (e.g., first lumen 24) extending therethrough. The first end 121 of the gas supply tube 122 may terminate at or within the container 130 such that the first lumen is in fluid communication with the interior volume 140 of the container 130. In some cases, the gas supply tube 122 may pass through an opening 126 in the cap 127 and extend within and into the interior volume 140 of the container 130. In some cases, the opening 126 may include an air gap. In some cases, the opening 126 may include a rubber seal that the gas supply tube 122 may be configured to pass through. In some cases, the first end 121 of the gas supply tube 122 may be detachable, and may engage with the cap 127 of the container 130, for example, using a luer lock system. In some cases, the gas supply tube 122 and the cap 127 may be a single, monolithic piece.

The second end 123 of the gas supply tube 122 may be configured to engage with a first end 111 of the coupling mechanism 120. In some cases, the second end 123 of the gas supply tube 122 may engage with the first end 111 of the coupling mechanism 120, for example, via a hose barb mechanism. In some cases, the second end 123 of the gas supply tube 122 may engage with the first end 111 of the coupling mechanism 120 via interference fit, a luer lock system, a luer slip system, or any other suitable type of engagement. The second end 112 of the coupling mechanism 120 may be configured to engage with an outlet 110 of the first gas supply 150. In some cases, the second end 112 of the coupling mechanism 120 may include a size and shape configured to facilitate an interference fit between the coupling mechanism 120 and the outlet 110 of the first gas supply 150.

A water supply tube 132 may include a first end 131, a second end 133, and a second lumen extending therethrough (e.g., second lumen 34). The water supply tube 132 may be configured to terminate at or in a bottom portion of the container 130 such that the second lumen is in fluid communication with the interior volume 140 of the container 130. In some cases, when the water supply tube 132 terminates at the bottom portion of the container 130, the water supply tube 132 may include an inner tube (e.g., inner tube 29) that may extend within and into the interior volume 140 of the container 130, which may contain the fluid 141. For example, the water supply tube 132 may act as a lens wash supply tube and the inner tube (e.g., inner tube 29) may act as an irrigation supply tube, or the water supply tube 132 may act as an irrigation supply tube and the inner tube may act as a lens wash supply tube, among other possibilities. In some cases, the water supply tube 132 may pass through an opening in the bottom portion of the container 130 and extend within and into the interior volume 140 of the container 130. In some cases, the opening in the bottom portion of the container may include a rubber seal that the water supply tube 132 may be configured to pass through. In some cases, the first end 131 of the water supply tube 132 may be detachable, and may engage with the container 130 using a luer lock system, and the inner tube may then extend through the opening in the bottom portion of the container 130 and into the interior volume 140 of the container 130. In some cases, the water supply tube 132 and the container 130 may be a single, monolithic piece. In some cases, the second end 133 of the water supply tube 132 may include a connector 135 configured to engage with a lens wash tube, an irrigation supply tube, or a split junction that may be configured to engage with both a lens wash tube and an irrigation supply tube.

When the coupling mechanism 120 is coupled to the outlet 110 of the first gas supply 150 and the gas supply tube 122, gas (e.g., air) is allowed to flow from the first gas supply 150 through the gas supply tube 122 and into the container 130. This allows the gas (e.g., air) to pressurize the fluid 141 (e.g., water) within the container 130 and force the fluid 141 out through the water supply tube 132 and through a lens wash tube and/or an irrigation supply tube.

In some cases, when the water supply tube 132 is connected to a lens wash tube, the volume of the flow rate of the lens wash is governed by gas pressure in the container 130. When gas pressure begins to drop in the container 130, as water is pushed out of the container 130 through the water supply tube 132, the first gas supply 150 replaces lost air supply in the container 130 to maintain a substantially constant pressure, which in turn provides for a substantially constant lens wash flow rate. In some embodiments, a filter (not shown) may be placed in the path of the gas supply tube 122 to filter-out undesired contaminants or particulates from passing into the container 130. In some embodiments, outflow check valves or other one-way valve configurations (not shown) may be placed in the path of the water supply tube 132 to help prevent water from backflowing into the container 130 after the water has passed the valve.

In some cases, a relatively higher flow rate of irrigation water is typically required compared to lens wash, since a primary use is to clear the treatment area in the patient of debris that obstructs the user's field of view. In some cases, irrigation may be achieved with the use of a pump (e.g., peristaltic pump). In some cases, irrigation may be achieved by adjusting the gas flow from the first gas supply 150 into the container 130. In some cases, in order to equalize the pressure in the container 130 as water is pumped out of the water supply tube 132 and subsequently the irrigation supply tube, an air vent (not shown) may be included in the cap 127 of the container 130. The vent allows atmospheric air into the water source preventing negative pressure build-up in the water source, which could create a vacuum that suctions undesired matter from the patient back through the endoscope toward the water source. In some embodiments, outflow check valves or other one-way valve configurations (not shown) may be placed in the path of the irrigation supply tube to help prevent back-flow into the container 130 after water has passed the valve.

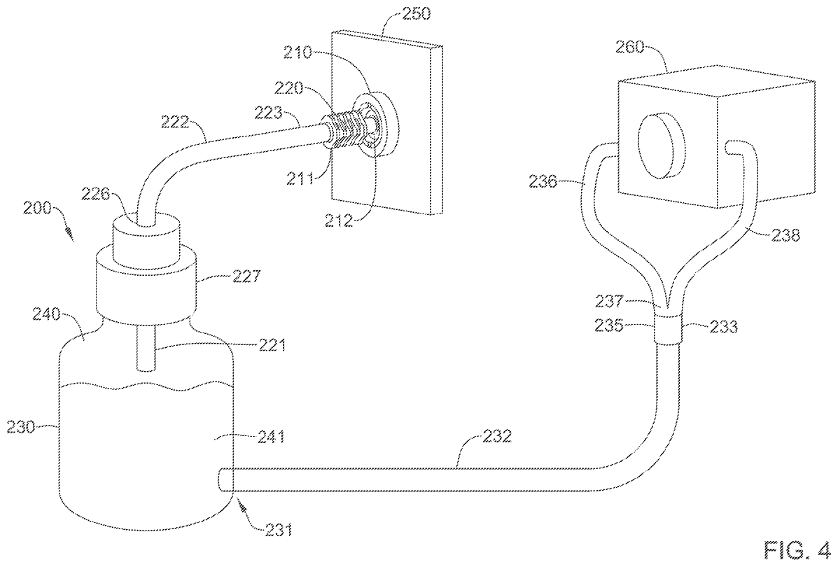

FIG. 4 illustrates an exemplary endoscope system including a container and tube set 200 coupled to an exemplary second gas supply 250, a lens wash tube 236, and an irrigation supply tube 238. The container and tube set 200 may be considered as an example of the container and tube set 10, as in FIG. 1. In some cases, the second gas supply 250 may be a CO₂ (carbon dioxide) source, such as a wall-based CO₂ outlet, as shown in FIG. 4. In some cases, the second gas supply 250 may be a portable CO₂ cylinder coupled to a CO₂ regulator. The second gas supply 250 may be configured to pump CO₂ through a gas supply tube 222 to a container 230. As shown in FIG. 4, the gas supply tube 222 may include may include a first end 221 and a second end 223. While not shown, the gas supply tube 222 may include a first lumen (e.g., first lumen 24) extending therethrough. The first end 221 of the gas supply tube 222 may terminate at or within the container 230 such that the first lumen is in fluid communication with an interior volume 240 of the container 230. In some cases, the gas supply tube 222 terminates at a cap 227 of the container 230. In some cases, the gas supply tube 222 may pass through an opening 226 in the cap 227 and extend within and into the interior volume 240 of the container 230. In some cases, the opening 226 may include an air gap. In some cases, the opening 226 may include a rubber seal that the gas supply tube 222 may be configured to pass through. In some cases, the first end 221 of the gas supply tube 222 may be detachable, and may engage with the cap 227 of the container 230, for example, using a luer lock system. In some cases, the gas supply tube 222 and the cap 227 may be a single, monolithic piece.

The second end 223 of the gas supply tube 222 may be configured to engage with a first end 211 of the coupling mechanism 220. In some cases, the second end 223 of the gas supply tube 222 may engage with the first end 211 of the coupling mechanism 220, for example, via a luer lock system 225. In some cases, the second end 223 of the gas supply tube 222 may engage with the first end 211 of the coupling mechanism 220 via interference fit, a luer slip system, or any other suitable type of engagement. The second end 212 of the coupling mechanism 220 may be configured to engage with an outlet 210 of the second gas supply 250. In some cases, the second end 212 of the coupling mechanism 220 may include a size and shape configured to facilitate an interference fit between the coupling mechanism 220 and the outlet 210 of the second gas supply 250.

A water supply tube 232 may include a first end 231, a second end 233, and a second lumen extending therethrough (e.g., second lumen 34). The water supply tube 232 may be configured to terminate at or in a bottom portion of the container 230 such that the second lumen is in fluid communication with the interior volume 240 of the container 230. In some cases, when the water supply tube 232 terminates at the bottom portion of the container 130, the water supply tube 232 may include an inner tube (e.g., inner tube 29) that may extend within and into the interior volume 240 of the container 230, which may contain the fluid 241. For example, the water supply tube 232 may act as a lens wash supply tube and the inner tube 29 may act as an irrigation supply tube, or the water supply tube 232 may act as an irrigation supply tube and the inner tube 29 may act as a lens wash supply tube, among other possibilities. In some cases, the water supply tube 232 may pass through an opening in the bottom portion of the container 230 and extend within and into the interior volume 240 of the container 230. In some cases, the opening in the bottom portion of the container may include a rubber seal that the water supply tube 232 may be configured to pass through. In some cases, the first end 231 of the water supply tube 232 may be detachable, and may engage with the container 230, for example, using a luer lock system, and the inner tube may then extend through the opening in the bottom portion of the container 230 and into the interior volume 240 of the container 230. In some cases, the water supply tube 232 and the container 230 may be a single, monolithic piece.

In some cases, the second end 233 of the water supply tube 232 may include a connector 235 configured to engage with a split junction 237 that may be configured to engage with both the lens wash tube 236 and the irrigation supply tube 238. The lens wash tube 236 and the irrigation supply tube 238 may both be configured to engage with an endoscope system 260. When the coupling mechanism 220 is coupled to the outlet 210 of the second gas supply 250 and the gas supply tube 222, gas (e.g., CO₂) is allowed to flow from the second gas supply 250 through the gas supply tube 222 and into the container 230. This allows the gas (e.g., CO₂) to pressurize the fluid 241 (e.g., water) within the container 230 and force the fluid 241 out through the water supply tube 232 and through the lens wash tube 236 and/or the irrigation supply tube 238 to the endoscope system 260.

In some cases, when the water supply tube 232 is connected to the lens wash tube 236, the volume of the flow rate of the lens wash is governed by gas pressure in the container 230. When gas pressure begins to drop in the container 230, as water is pushed out of the container 230 through the water supply tube 232, the second gas supply 250 replaces lost CO₂ supply in the container 230 to maintain a substantially constant pressure, which in turn provides for a substantially constant lens wash flow rate. In some embodiments, a filter (not shown) may be placed in the path of the gas supply tube 222 to filter-out undesired contaminants or particulates from passing into the container 230. In some embodiments, outflow check valves or other one-way valve configurations (not shown) may be placed in the path of the water supply tube 232 to help prevent water from backflowing into the container 230 after the water has passed the valve.

In some cases, a relatively higher flow rate of irrigation water is typically required compared to lens wash, since a primary use is to clear the treatment area in the patient of debris that obstructs the user's field of view. In some cases, irrigation may be achieved with the use of a pump (e.g., peristaltic pump). In some cases, irrigation may be achieved by adjusting the gas flow from the second gas supply 250 into the container 230. In some cases, in order to equalize the pressure in the container 230 as water is pumped out of the water supply tube 232 and subsequently the irrigation supply tube 238, an air vent (not shown) may be included in the cap 227 of the container 230. The vent allows atmospheric air into the water source preventing negative pressure build-up in the water source, which could create a vacuum that suctions undesired matter from the patient back through the endoscope system 260 toward the water source. In some embodiments, outflow check valves or other one-way valve configurations (not shown) may be placed in the path of the irrigation supply tube to help prevent back-flow into the container 230 after water has passed the valve.

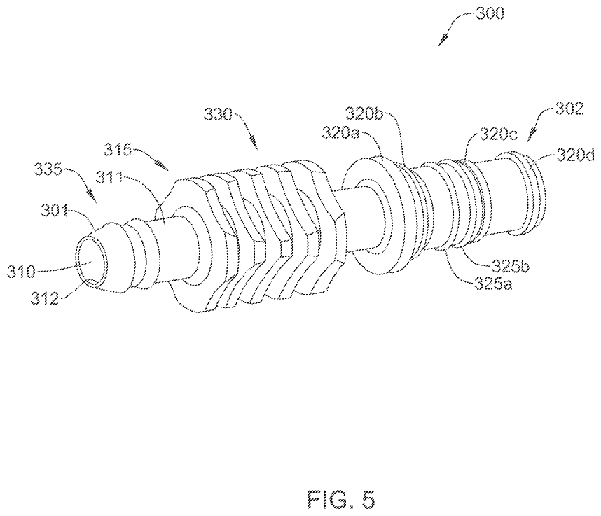

FIG. 5 illustrates a perspective view of an exemplary coupling mechanism 300. The coupling mechanism 300 may be considered as an example of the coupling mechanism 20, 120, 220 as in FIG. 1, FIG. 2, FIG. 3, respectively. The coupling mechanism 300 may include a substantially tubular body 315 having an outer surface 311, an inner surface 312, a first end 301 and a second end 302. A lumen 310 may extend through the tubular body 315 from the first end 301 to the second end 302. In some cases, the first end 301 of the tubular body 315 may be configured to engage with a second end of a gas supply tube (e.g., gas supply tube 22, 122, 222), for example, via a hose barb mechanism. In such cases, a hose barb 335, may be positioned at the first end 301 of the tubular body 315. In some cases, the coupling mechanism 300 may include a grip 330 which a user may hold onto while connecting the coupling mechanism 300 to the second end of a gas supply tube and/or to a gas supply. Preferred materials for the tubular body 315 include polycarbonate materials. Where polymers are selected as tubular body 315 materials, the materials may have a durometer in a range of about 75A to 90A Shore hardness, among other possible values.

The second end 302 of the tubular body 315 may include a plurality of ridges 320a, 320b, 320c, 320d on the outer surface 311 of the tubular body 315. In some cases, an annular seal 325 may be positioned within the plurality of ridges 320a, 320b, 320c, 320d (between ridges 320b, 320c in the embodiment shown). In some cases, the annular seal 325 may be overmolded onto the second end 302 of the tubular body 315. Preferred materials for the annular seal 325 include thermoplastic elastomers (TPE), silicone, or any other suitable material. Where polymers are selected as annular seal 325 materials, the materials may have durometer in a range of about 40A to 60A Shore hardness, among other possible values.

The plurality of ridges 320a, 320b, 320c, 320d, 325a, 325b may include a size and shape configured to facilitate an interference fit between the coupling mechanism 300 and a first gas supply (e.g., first gas supply 150) and a second gas supply (e.g., second gas supply 250) interchangeably. The plurality of ridges 320a, 320b, 320c, 320d may include a at least a first ridge (e.g., ridge 320a) of the plurality of ridges 320a, 320b, 320c, 320d having a size and shape configured to engage with the first gas supply, such that a seal is formed between the coupling mechanism 300 and the first gas supply and at least a second ridge (e.g., 320d) of the plurality of ridges 320a, 320b, 320c, 320d having a size and shape configured to engage with the second gas supply, such that a seal is formed between the coupling mechanism 300 and the second gas supply. For example, at least the first ridge (e.g., ridge 320a) may have an outer diameter that is larger than the remaining ridges of the plurality of ridges 320a, 320b, 320c, 320d. While it is shown that the plurality of ridges 320a, 320b, 320c, 320d, 325a, 325b includes six ridges, it may be contemplated that the plurality of ridges 320 includes four ridges, five ridges, seven ridges, eight ridges, or any other suitable number of ridges as desired.

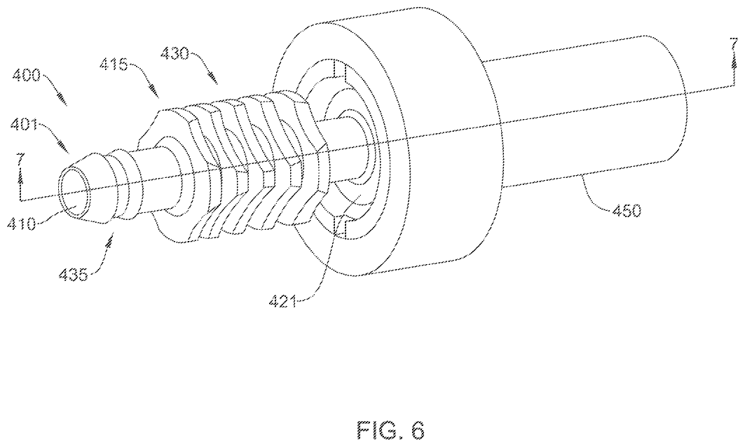

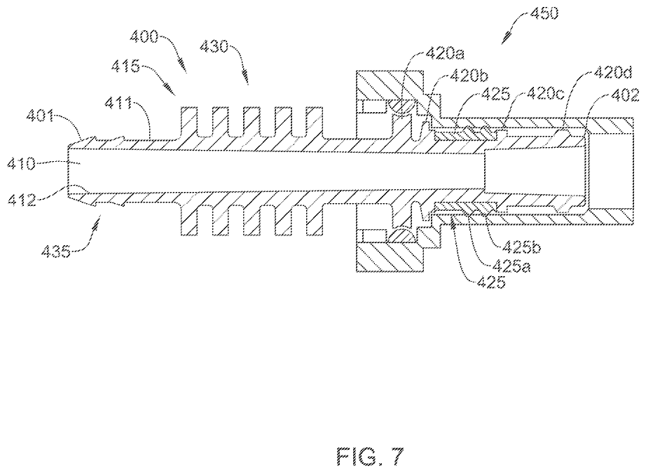

FIG. 6 illustrates an exemplary coupling mechanism 400 coupled to an exemplary outlet 450 of a first gas supply (e.g., first gas supply 150). FIG. 7 illustrates a cross-section view of the exemplary coupling mechanism 400 coupled to the exemplary outlet 450 of the first gas supply of FIG. 6, taken at line 7-7. The coupling mechanism 400 may be considered as an example of the coupling mechanism 300, as in FIG. 5. As shown in FIGS. 6 to 7, The coupling mechanism 400 may include a substantially tubular body 415 having an outer surface 411, an inner surface 412, a first end 401 and a second end 402. A lumen 410 may extend through the tubular body 415 from the first end 401 to the second end 402. In some cases, the first end 401 of the tubular body 415 may be configured to engage with a second end of a gas supply tube (e.g., gas supply tube 22), for example, via a hose barb or another suitable mechanism. In such cases, a hose barb 435, may be positioned at the first end 401 of the tubular body 415. In some cases, the coupling mechanism 400 may include a grip 430 which a user may hold onto while connecting the coupling mechanism 400 to the second end of a gas supply tube and/or to a gas supply. Preferred materials for the body include polycarbonate materials. Where polymers are selected as tubular body 415 materials, the materials may have durometer in a range of about 75A to 90A Shore hardness, among other possible values.

The second end 402 of the tubular body 415 may include a plurality of ridges 420a, 420b, 420c, 420d, on the outer surface 411 of the tubular body 415. In some cases, an annular seal 425 may be positioned between the plurality of ridges (between ridges 420b, 420c in the embodiment shown). The annular seal 425 may include one or more ridges 425a, 425b. In other embodiments (not shown), one or more ridges may be created by the surfaces of one or more O-rings. In some cases, the annular seal 425 may be overmolded onto the second end 402 of the tubular body 415. Preferred materials for the annular seal 425 include thermoplastic elastomers (TPE), silicone, or any other suitable material. Where polymers are selected as annular seal 425 materials, the materials may have durometer in a range of about 40A to 60A Shore hardness, among other possible values.

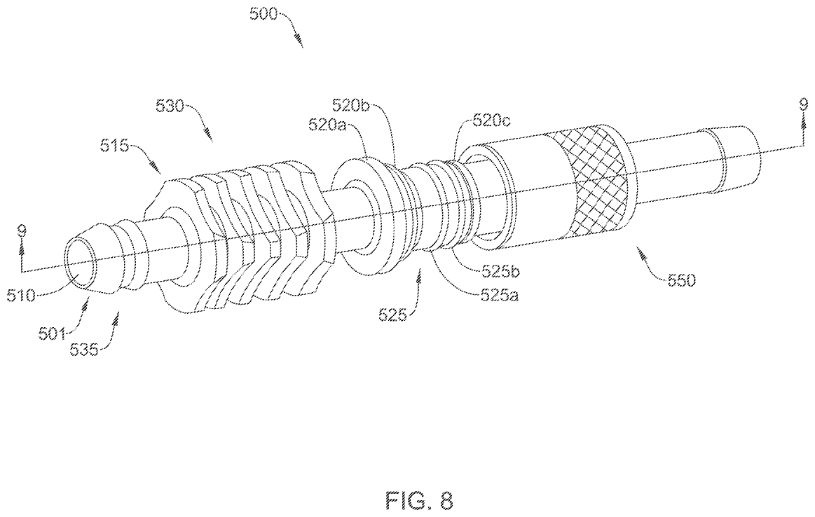

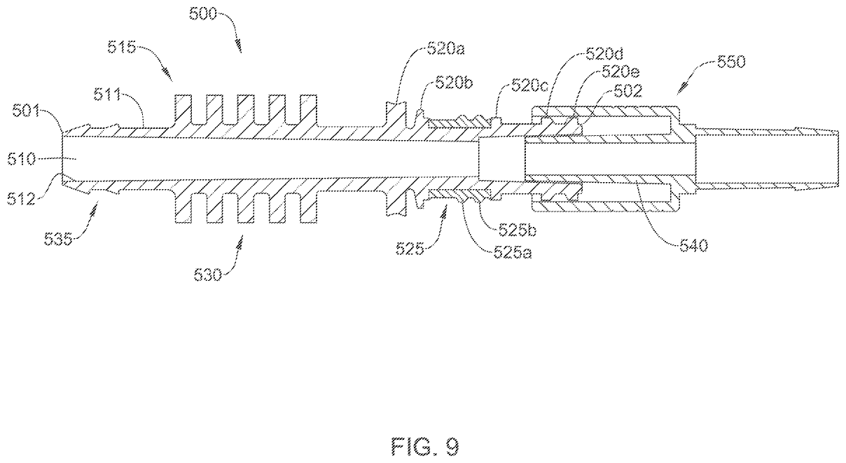

The plurality of ridges 420a, 420b, 420c, 420d, 425a, 425b may include a size and shape configured to facilitate an interference fit between the coupling mechanism 400 and a first gas supply (e.g., first gas supply 150) and a second gas supply (e.g., second gas supply 250) interchangeably. The plurality of ridges 420a, 420b, 420c, 420d may include a at least a first ridge (e.g., ridge 420a) of the plurality of ridges 420a, 420b, 420c, 420d having a size and shape configured to engage with the first gas supply, such that a seal is formed between the coupling mechanism 400 and the first gas supply. For example, at least the first ridge (e.g., ridge 420a) may have an outer diameter that is larger than the remaining ridges of the plurality of ridges 420a, 420b, 420c 420d. As shown in FIG. 7, the first ridge 420d may include a size and shape configured to engage with the outlet 450 of the first gas supply, such that a seal is formed between the coupling mechanism 400 and the outlet 450 of the first gas supply. Moreover, the ridges 425a, 425b, of the annular seal 425 may include a size and shape configured to engage with the outlet 450 of the first gas supply, such that an additional seal is formed between the coupling mechanism 400 and the outlet 450 of the first gas supply. Although not shown, ridges 420c, 420d are configured to engage with the outlet of the second gas supply, analogous to the ridges 520d, 520e in FIGS. 8 to 9. While it is shown that the plurality of ridges 420a, 420b, 420c, 420d, 425a, 425b includes six ridges, it may be contemplated that the plurality of ridges 420a, 420b, 420c, 420d, 425a, 425b includes three, four ridges, seven ridges, eight ridges, or any suitable number of ridges as desired.

FIG. 8 illustrates an exemplary coupling mechanism 500 coupled to an exemplary outlet 550 of a second gas supply (e.g., second gas supply 250). FIG. 9 illustrates a cross-section view of the exemplary coupling mechanism 500 coupled to the exemplary outlet 550 of the second gas supply of FIG. 8, taken at line 9-9. The coupling mechanism 500 may be considered as an example of the coupling mechanism 300, as in FIG. 5. The coupling mechanism 500 represents a slight variation from coupling mechanism 400 as it includes an additional ridge as discussed below. As shown in FIGS. 8 to 9, the coupling mechanism 500 may include a substantially tubular body 515 having an outer surface 511, an inner surface 512, a first end 501 and a second end 502. A lumen 510 may extend through the tubular body 515 from the first end 501 to the second end 502. In some cases, the first end 501 of the tubular body 515 may be configured to engage with a second end of a gas supply tube (e.g., gas supply tube 22), for example, via a hose barb mechanism. In such cases, a hose barb 535, may be positioned at the first end 501 of the tubular body 515. In some cases, the coupling mechanism 500 may include a grip 530 which a user may hold onto while connecting the coupling mechanism 500 to the second end of a gas supply tube and/or to a gas supply. Preferred materials for the tubular body 515 include polycarbonate materials. Where polymers are selected as tubular body 515 materials, the materials may have durometer in a range of about 75A to 90A Shore hardness, among other possible values.

The second end 502 of the tubular body 515 may include a plurality of ridges 520a, 520b, 520c, 520d, 520e on the outer surface 511 of the tubular body 515. In some cases, an annular seal 525 may be positioned within the plurality of ridges 520a, 520b, 520c, 520d, 520e (between ridges 520b, 520c in the embodiment shown). The annular seal 525 may include one or more ridges 525a, 525b. In other embodiments (not shown), one or more ridges may be created by the surfaces of one or more O-rings. In some cases, the annular seal 525 may be overmolded onto the second end 502 of the tubular body 515. The plurality of ridges 520a, 520b, 520c, 520d, 520e, 525a, 525b may include a size and shape configured to facilitate an interference fit between the coupling mechanism 500 and a first gas supply (e.g., first gas supply 150) and a second gas supply (e.g., second gas supply 250) interchangeably. In some cases, the ridges 520d, 520e of the plurality of ridges 520a, 520b, 520c, 520d, 520e, 525a, 525b may be configured to engage with the outlet 550 of the second gas supply. The ridges 520d, 520e may engage with the outlet 550 via an interference fit or a transition fit. Preferred materials for the annular seal 525 include thermoplastic elastomers (TPE), silicone, or any other suitable material. Where polymers are selected as annular seal 525 materials, the materials may have durometer in a range of about 40A to 60A Shore hardness, among other possible values.

In some cases, the second end 502 of the coupling mechanism 500 may be inserted into the second gas supply, which may be a wall-based CO₂ outlet. The second end 502 of the coupling mechanism 500 may be plugged into the outlet 550 of the second gas supply, and a valve 540 within the outlet 550 may extend within the lumen 510 of the coupling mechanism 500. The ridges 520d, 520e of the second end 502 work together to provide an interference fit to hold the coupling mechanism 500 tight within the outlet 550 of the second gas supply. Further, the plurality of ridges 520a, 520b, 520c, 520d, 520e, 525a, 525b may include a first ridge 520a that may have an outer diameter that is larger than the remaining ridges of the plurality of ridges 520b, 520c, 520d, 520e, 525a, 525b. Although not shown, ridges 520a, 525a, 525b may include a size and shape configured to engage with the outlet of the first gas supply, such that a seal is formed between the coupling mechanism 500 and the outlet of the first gas supply, analogous to the ridges 420a, 425a, 425b in FIGS. 6 to 7. While it is shown that the plurality of ridges includes seven ridges 520a, 520b, 520c, 520d, 520e, 525a, 525b, it may be contemplated that the plurality of ridges 520 includes three, four ridges, six ridges, eight ridges, or any suitable number of ridges as desired.

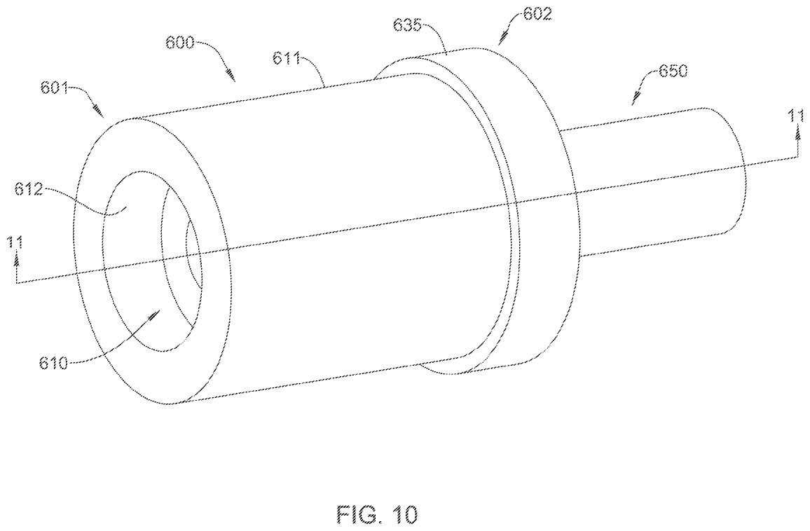

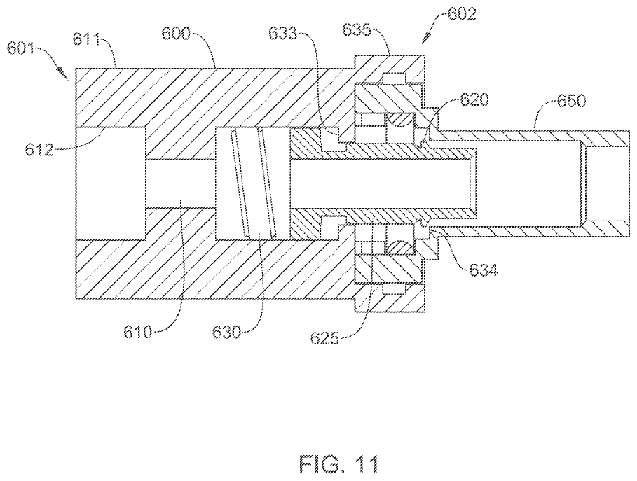

FIG. 10 illustrates an exemplary coupling mechanism 600 coupled to an exemplary outlet 650 of a gas supply (e.g., the first gas supply 150). FIG. 11 illustrates a cross-section view of the exemplary coupling mechanism 600 coupled to the exemplary outlet 650 of the gas supply of FIG. 10, taken at line 11-11. As shown in FIGS. 10 to 11, the coupling mechanism 600 may include an outer surface 611, an inner surface 612, a first end 601 and a second end 602. A lumen 610 may extend through the coupling mechanism 600 from the first end 601 to the second end 602. In some cases, the first end 601 of the coupling mechanism 600 may be configured to engage with a second end of a gas supply tube (e.g., gas supply tube 22), for example, via a hose barb mechanism, a luer lock system, or any other suitable connection. Preferred materials for the coupling mechanism 600 include polycarbonate materials. Where polymers are selected as coupling mechanism 600 materials, the materials may have durometer in a range of about 75A to 90A Shore hardness, among other possible values.

The second end 602 of the coupling mechanism 600 may include an outer lip 635. In some cases, the outer lip 635 may be configured to fit over an outer surface of an outlet 650 of a gas supply (e.g., first gas supply 150). The outer lip 635 of the coupling mechanism 600 may include a size and shape configured to provide an interference fit with the outlet 650 of the gas supply. This interference fit holds the coupling mechanism 600 tight onto the outlet 650 of the gas supply to prevent the coupling mechanism 600 from backing off of the outlet 650. In some cases, the coupling mechanism 600 may include a ridge 620 on a male portion 625 of the coupling mechanism 600. The ridge 620 may create an additional seal between the coupling mechanism 600 and the outlet of the gas supply 650.

The coupling mechanism 600 may include a spring 630 attached to the male portion 625 within the coupling mechanism 600. The spring 630 may be positioned around the lumen 610 such that a gas (e.g., air, CO₂) may pass from the outlet 650 of the gas supply through the lumen 610 of the coupling mechanism 600 and into a gas supply tube. The spring 630 may be configured to move along an axis of the coupling mechanism 600 such that the ridge 620 of the male portion 625 may engage with stop 634 of the outlet 650 of the gas supply (similar to a coaxial cable). In other embodiments, an internal stop 633 may prevents the male portion 625 from moving too far distally from the coupling mechanism 600.

In some cases, the male portion 625 of the coupling mechanism 600 may be inserted into a second gas supply (not shown), which may be a wall-based CO₂ outlet like outlet 550 shown in FIGS. 8-9. The male portion 625 of the coupling mechanism 600 may be plugged into the outlet (e.g., outlet 550) of the second gas supply, and a valve 540 within the outlet may extend within the lumen 610 of the coupling mechanism 600. The ridge 620 may work to provide an interference fit to hold the coupling mechanism 600 tight within the outlet of the second gas supply.

In addition to the preferred materials described above, coupling mechanism 20, 120, 220, 300, 400, 500, 600 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A container and tube set arranged and configured to couple to an endoscope, a first gas supply, and a second gas supply for use in an endoscopic procedure, the container and tube set comprising:
a container having an interior volume configured to contain a fluid;
a gas supply tube including a first end, a second end, and a first lumen extending therethrough, wherein the first end of the gas supply tube terminates at or in the container and the first lumen is in fluid communication with the interior volume of the container;
a coupling mechanism having a first end and a second end, wherein the first end of the coupling mechanism is configured to engage with the second end of the gas supply tube and the second end of the coupling mechanism is configured to engage with both the first gas supply and the second gas supply interchangeably;
a water supply tube including a first end, a second end, and a second lumen extending therethrough, wherein the first end of the water supply tube terminates at or in a bottom portion of the container, the second lumen is in fluid communication with the interior volume of the container, and the second end of the water supply tube is positioned external to the container.

2. The container and tube set of claim 1, further comprising a split flow junction at the second end of the water supply tube wherein the second lumen of the water supply tube is in fluid communication with both a third lumen of a lens wash tube and a fourth lumen of an irrigation supply tube.

3. The container and tube set of claim 1, wherein the second end of the coupling mechanism includes a plurality of ridges on the outer surface of the coupling mechanism.

4. The container and tube set of claim 3, wherein an annular seal comprising at least one of the plurality of ridges is positioned between two others of the plurality of ridges.

5. The container and tube set of claim 3, wherein the plurality of ridges includes at least a first ridge of the plurality of ridges having a size and shape configured to engage with the first gas supply, such that a seal is formed between the coupling mechanism and the first gas supply and at least a second ridge of the plurality of ridges having a size and shape configured to engage with the second gas supply, such that a seal is formed between the coupling mechanism and the second gas supply.

6. The container and tube set of claim 1, wherein the first end of the coupling mechanism includes a hose barb mechanism.

7. The container and tube set of claim 1, wherein the coupling mechanism is a single, monolithic structure.

8. The container and tube set of claim 1, wherein the second end of the coupling mechanism is shaped to facilitate an interference fit between the coupling mechanism and the first gas supply and the second gas supply interchangeably.

9. The coupling mechanism of claim 1, wherein the first gas supply is a processor capital configured to pump air through the gas supply tube and wherein the second gas supply is a carbon dioxide (CO₂) source configured to pump CO₂ through the gas supply tube.

10. A coupling mechanism for an endoscope system comprising:
a substantially tubular body having an outer surface, and inner surface, a first end, and a second end; and
a lumen extending through the tubular body from the first end to the second end;
wherein the first end is configured to engage with a gas supply tube and the second end is configured to engage with both a first gas supply and a second gas supply interchangeably; and
wherein the gas supply tube is configured to engage with a container configured to contain a fluid.

11. The coupling mechanism of claim 10, wherein the second end of the coupling mechanism includes a plurality of ridges on the outer surface of the coupling mechanism.

12. The coupling mechanism of claim 11, wherein an annular seal comprising at least one of the plurality of ridges is positioned between two others of the plurality of ridges.

13. The coupling mechanism of claim 11, wherein the plurality of ridges includes at least a first ridge of the plurality of ridges having a size and shape configured to engage with the first gas supply, such that a seal is formed between the coupling mechanism and the first gas supply and at least a second ridge of the plurality of ridges having a size and shape configured to engage with the second gas supply, such that a seal is formed between the coupling mechanism and the second gas supply.

14. The coupling mechanism of claim 10, wherein the first end of the coupling mechanism includes a hose barb mechanism.

15. The coupling mechanism of claim 10, wherein the first gas supply is a processor capital configured to pump air through the gas supply tube.

16. The coupling mechanism of claim 10, wherein the second gas supply is a carbon dioxide (CO₂) source configured to pump CO₂ through the first gas supply tube.

17. A coupling mechanism for an endoscope system comprising:
a substantially tubular body having an outer surface, and inner surface, a first end, and a second end;
a lumen extending through the tubular body from the first end to the second end;
a hose barb mechanism positioned at the first end of the tubular body; and
a plurality of ridges positioned at an outer surface of the second end of the tubular body;
wherein an annular seal is positioned within the plurality of ridges;
wherein the first end is configured to engage with a second end of a gas supply tube and the second end is configured to engage with both a first gas supply and a second gas supply interchangeably; and
wherein a first end of the gas supply tube is configured to engage with a container configured to contain a fluid.

18. The coupling mechanism of claim 17, wherein the first gas supply is a processor capital configured to pump air through the gas supply tube, and the second gas supply is a carbon dioxide (CO₂) source configured to pump CO₂ through the gas supply tube.

19. The coupling mechanism of claim 17, wherein the plurality of ridges includes at least a first ridge of the plurality of ridges having a size and shape configured to engage with the first gas supply, such that a seal is formed between the coupling mechanism and the first gas supply and at least a second ridge of the plurality of ridges having a size and shape configured to engage with the second gas supply, such that a seal is formed between the coupling mechanism and the second gas supply.

20. The coupling mechanism of claim 17, wherein the second end of the coupling mechanism is shaped to facilitate an interference fit between the coupling mechanism and the first gas supply and the second gas supply interchangeably.
